(19) United States
(12) Patent Application Publication
(10) Pub. No.: US 2019/0135737 A1
(43) Pub. Date: May 9, 2019

(54) METHOD FOR PRODUCING XYLYLENE DIISOCYANATE (XDI)

(71) Applicant: VENCOREX FRANCE, SAINT-PRIEST (FR)

(72) Inventors: Anne-Marie RATABOUL-LEDUC, LYON (FR); Jean-Marie BERNARD, SAINT LAURENT D'AGNY (FR); Julien TOURNAYRE, OULLINS (FR)

(21) Appl. No.: 16/091,586

(22) PCT Filed: Apr. 7, 2017
(86) PCT No.: PCT/EP2017/058341

§ 371 Date: Oct. 5, 2018

(30) Foreign Application Priority Data
Apr. 7, 2016 (FR) 1653051

(51) Int. Cl. C07C 263/10; C07C 263/20

(52) U.S. Cl. CPC C07C 263/10; C07C 265/14; C07C 263/20

(57) ABSTRACT

Disclosed is to a method for producing xylylene diisocyanate (XDI), in particular meta-xylylene diisocyanate (mXDI), including the following steps: a) phosgenation of xylylene-diamine (XDA), in particular m-xylylene-diamine (mXDA) in the case of mXDI; b) eliminating the hydrochloric acid from the reaction medium obtained in step (a) at a temperature of between 120 and 190° C. and a pressure of between 1 mbar and 20 bar.

### TECHNICAL FIELD

The invention relates to a process for preparing xylylene diisocyanates of the formula R(NCO)₂ (where R is the dimethylbenzene nucleus), abbreviated as XDI, particularly meta-xylylene diisocyanate, abbreviated as mXDI, by phosgenation of the corresponding xylylene diamine (or XDA). The invention also relates to the product XDI thus obtained. The XDI can also be prepared without phosgenation, for example from xylylene dicarbamate, xylylene diformamide or xylylene dihalide. These last types of preparation do not fall within the field of the present invention.

The XDI is an araliphatic diisocyanate whose properties are intermediate between the (cyclo)aliphatic diisocyanates—such as hexamethylene diisocyanate (HDI) or isophorone diisocyanate (IPDI)—and the aromatic diisocyanates—such as toluene diisocyanate (TDI) or diphenylmethane diisocyanate (MDI). In this text, mXDI also refers to ortho- and para-xylylene diisocyanate, unless otherwise indicated, and therefore generally refers to XDI.

The polycondensation of diisocyanates with polyols, polyamines or polythiols occurs in the realization of polyurethane, polyurea and polythiourethane products more specifically in relation to the field of paints, adhesives, transparent materials or optical glass. In the latter case, optical glass made from MXDI has a particularly high refractive index allowing for the production, with equivalent performance, of thinner lenses (or for the production of lenses with superior performance and equal thickness).

For a given application, an isocyanate is selected depending on the properties sought and the desired reactivity. While (cyclo)aliphatic diisocyanates (HDI, IPDI) have a high level of resistance to yellowing associated with low reactivity, aromatic diisocyanates (TDI, MDI) are substantially more reactive but quickly turn yellow. XDI, and in particular mXDI, are concerned firstly with having a resistance to yellowing that is superior to that of aromatic isocyanates and, secondly, with having isocyanate functional groups with a reactivity that is intermediate between those of aliphatic and aromatic isocyanates. These properties ensure a reactivity and a useful life compatible with certain targeted applications such as food packaging film. In fact, for these applications, a limited useful life, of 6 months for example, is quite compatible with the useful life of their use.

Conventionally, XDI, like other diisocyanates, is prepared by reacting phosgene COCl₂ with a diamine, here a xylylene diamine R(NH₂)₂, for example m-xylylenediamine to prepare mXDI. This phosgenation reaction leads to the conversion of the diamine and is divided into two steps: a step of forming intermediate products, particularly carbamyl chlorides, followed by a step involving the transformation of said intermediate products to isocyanate—at a reaction temperature typically in the range 130-170° C.—and at a pressure between atmospheric pressure and a pressure of about 4 bar.

Thus, in a simplified way, a first step can produce mainly carbamyl chlorides and hydrochloric acid by reaction of amino functions with phosgene according to the reaction:

R(NH₂)₂+2COCl₂→R(NHCOCl)₂+2HCl (I)

and the second step provides XDI by decomposition of carbamyl chlorides according to the following dehydrochlorination reaction:

R(NHCOCl)₂→R(NCO)₂+2 HCl (II)

After purification, the yield of XDI is between 91 and 95%.

### State of the Art

It is possible to achieve the phosgenation by different routes, depending on the conditions of temperature and pressure, whether in a completely liquid, partly liquid and partly gaseous, or completely gaseous phase. According to these conditions, intermediate products consist of mixtures of various amine hydrochlorides and/or different carbamyl chlorides in varying proportions.

In particular, better reactivity can be obtained for the preparation of isocyanates by a prior step of hydrochlorination of mXDA diamine by reaction with hydrochloric acid. The intermediate product obtained—the corresponding amine hydrochloride R(NH₂.HCl)₂—is then subjected, in the second step of phosgenation, to an elevated reaction temperature, for example 190° C., as described for example in U.S. Pat. No. 5,523,467.

According to another example, the process described in CN 102070491 provides a step of forming a salt by hydrochlorination of XDA solution in an inert solvent mixture of hydrochloric acid. This step is followed by a step involving the concentration by centrifugation of the hydrochloride formed, then steps of high and low pressure phosgenation. In this process, the hydrochlorination reaction of the amine is maintained at low concentration in solution and the hydrochloride is concentrated to prepare the XDI. This process ensures a high conversion rate from an amine hydrochloride solution.

Further improvements were obtained by special treatment. The use of a reflux of dichlorobenzene allows for a reduction in the reaction temperature to 120-125° C. with a good yield (91.4%, for example) and a good degree of purity (99.3%, for example). Moreover, conducting the reaction under a nitrogen atmosphere improves efficiency (92.9%), as already cited in U.S. Pat. No. 5,523,467. In addition, a pressure increases of up to 2 bars during the reaction of hydrochloric acid with the mXDA diamine establishes an even better yield (98.8%) but a degree of purity that is substantially inferior (98.4%). However, this type of process is slow and requires a hydrochlorination step.

The phosgenation step is usually followed by a purification step which removes the reaction solvent as well as impurities to obtain the desired isocyanate compound with enhanced purity. Some of the species to be removed contain in particular hydrolyzable chlorine. The term “hydrolyzable chlorine” refers here to atoms of labile chlorine present in the isocyanate compounds, as defined for example in GB1350374.

It is also known that for some end applications, especially in the field of polyurethane foams, the presence of hydrolyzable chlorine in the isocyanate alters the properties of the final product.

In an attempt to reduce the amount of hydrolyzable chlorine of raw organic isocyanates, U.S. Pat. No. 3,219,678 proposes a high-temperature purification—at least equal to 190° C. in the examples—between two distillations to remove the solvent and residual impurities respectively. However, this solution provides high temperatures for aromatic isocyanates as these high temperatures may decompose araliphatic isocyanates, particularly XDI.

In order to remove the hydrolyzable chlorine, it is also possible to add targeted removal additives of metals and metal oxides, imidazoles, sulfonic acids and their esters, diethyl sulphate, sulfuric acid, trialkyl phosphates, or epoxy compounds in advance. Other additives—such as compounds having at least one NH group (urea, biuret, caprolactam, ammonium salts, carbodiimides, primary or secondary amine salts, etc.) or tertiary alcohols—have been proposed.

However, these additives generate a substantial decrease in the amount of isocyanate groups and an increase in viscosity, they substantially reduce the yield and generally require an additional separation of the isocyanate and the additive.

In addition, XDI isocyanates differentiate themselves from aromatic isocyanates, especially TDI, by the formation of monoisocyanate chloroalkyl compounds, particularly chloromethyl-benzyl-isocynate CIRNCO (hereinafter CIBi), and even dichloroxylene. The CIBi and its derivatives are derived from the substitution of an isocyanate function with a chlorine atom. These byproducts are particularly difficult to remove.

The CIBi and its derivatives are generally present in proportions of 3 to 10% or even 20%, as indicated in patent EP 0 384 463. The CIBi accelerates gelation of the prepolymer and affects the resin properties of the polyurethane obtained. This is why conditions preventing the formation of Clbi and its derivatives have been actively sought.

Research has thus focused on the conditions of phosgenation to reduce the amount of chlorinated byproducts. In U.S. Pat. No. 3,470,227, the phosgenation was conducted between 2 and 5 bar with an adjusting valve and a degassing of hydrochloric acid to maintain the pressure at a constant value.

However, if the phosgenation reaction temperature exceeds 180° C., to reduce the reaction time, there is a significant increase in byproducts. The use of an ester-type solvent such as hexyl acetate or amyl acetate was then recommended—for example in EP 0 384 463 cited above—to limit their formation.

Research has also focused on the purification conditions, including the reduction of CIBi content in XDI. Thus, to improve the separation of CIBi from XDI, an inert gas or a counter-current solvent may also be employed during distillation, as shown in FR 1 555 517, GB 1 119 459 or FR 1 555 515. The distillation temperature is thereby also reduced.

In general, it appears that, for the purification of XDI by distillation, the final step to remove light species is difficult because the main impurity to be removed, CIBi, has a vapor pressure close to that of XDI.

### DISCLOSURE OF THE INVENTION

The invention aims to develop an optimized process for preparing XDI by phosgenation of XDA amines through non hydrochloride (that is, the process of the invention does not include prior steps of preparing hydrochloride and does not include the implementation of species of hydrochloride) by proposing to minimize or eliminate the presence of intermediate chemicals while controlling the progress of the reaction, particularly by reducing the risk of thermal runaway, and to significantly increase the yield. The invention further proposes to facilitate the separation of compounds from the phosgenation, by avoiding catalysis of side reactions leading to heavy compounds, while reducing the amount of waste for disposal, in particular of heavy products.

To do this, the invention proposes to promote—before separation to eliminate the solvent—the transformation of intermediate chemicals formed during the phosgenation process by molecules having isocyanate functional groups with the release of HCl.

The term “intermediate chemical species” means species containing at least one —NH—CO—Cl function, referred to as a carbamyl chloride function, or at least one —N(—CO—Cl)—CO—NH— function, referred to as an allophanoyl chloride function and/or functions derived by combination or condensation of these species with each other and/or their combination and that can release at least one molecule of HCl. The carbamyl chlorides and allophanoyl chlorides functions are in balance with the following isocyanate functions.[sic] the following reactions:

(1) isocyanate+HCl Hcarbamyle chloride

(2) isocyanate+carbamyle chloride←allophanoyl chloride

By referring to K1 as the constant of the equilibrium of the reaction (1) for the mono- and di-carbamyl chloride of mXDI, TDI and HDI taken independently and K2 as the constant of the equilibrium of the reaction (2) for the allophanoyl chloride of mXDI, TDI and HDI taken independently, if one compares these different constants for different isocyanates (mXDI, TDI and HDI), it is clear that:

K1(mXDI)/K1(TDI)≈7

K1(mXDI)/K1(HDI)≈1

K2(mXDI)/K2(TDI)≈8

K2(mXDI)/K2(HDI)≈4

This shows that, unlike other isocyanates, allophanoyl chloride of mXDI is stable. There is therefore an interest in minimizing or even eliminating it as soon as possible to avoid having a negative impact on the final amount of mXDI. There is therefore an interest in eliminating or at least reducing the formation of these intermediate chemical species.

According to an advantageous feature, the intermediate chemical species transformed during the process according to the invention and having substantially the functions —NHCOCl carbamyl chloride of XDI, —N(—CO—Cl)—CO—NH— allophanoyl chloride of XDI and/or functions derived by combination or condensation of these species with each other, preferably have functions having one and/or the other of the formulas developed respectively of the form:

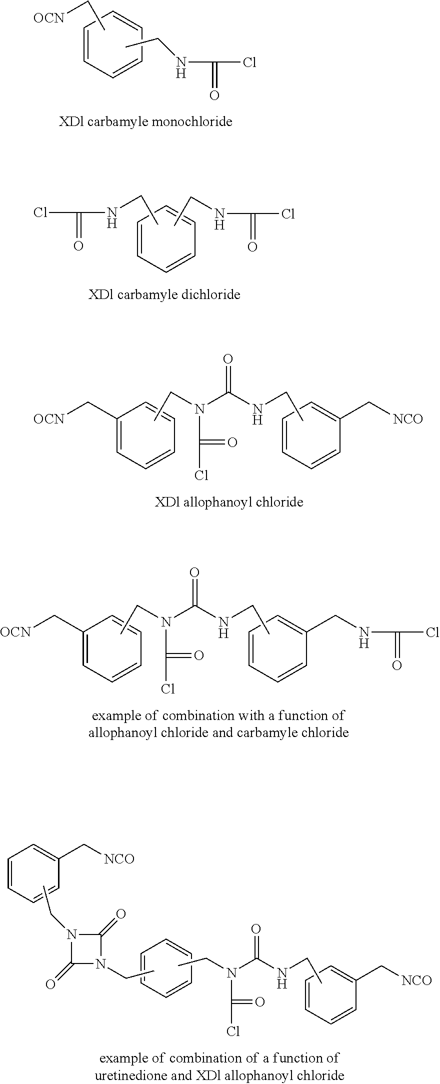

These intermediate chemical species may also contain other functions, such as the functions of uretidinedione (also called dimer), isocyanurate, carbodiimide, iminouretidinedione, iminotriazinedione and/or oxadiazinetrione groups, as well as their combination with the chlorides of carbamyl and allophanoyl.

The inventors have surprisingly found that the earlier the transformation of these intermediate chemical species occurs in the reaction process, the higher the yield of XDI. Furthermore, the inventors have also surprisingly found that the presence of intermediate chemical species persists at the end of the phosgenation reaction of XDA, after the removal of phosgene while, conventionally, for the aromatic or aliphatic diisocyanates, such as HDI, TDI or IPDI, such intermediate chemical species can be detected during the preparation but are virtually no longer present after completion of the phosgenation.

Furthermore, the inventors have observed, surprisingly, that the presence of intermediate chemical species significantly lowers the thermal runaway temperature threshold in the purification steps which follow removal of the solvent. Furthermore, the inventors found that the greater the amount of intermediate chemical species, the more the thermal decomposition temperature threshold is lowered. This causes a risk to safety during the purification of XDI.

The invention therefore proposes the removal of hydrochloric acid from the reaction medium as soon as possible under specific conditions allowing a controlled displacement of the reaction (1) to the isocyanate production, to produce in larger quantities the isocyanate compound sought and to limit the formation of intermediate chemical species.

The invention is more specifically aimed at a process for preparing XDI xylylene diisocyanates, particularly mXDI meta-xylylene diisocyanates, comprising the steps of:
a. phosgenation of the xylylene diamine XDA, in particular m-xylylene diamine mXDA in the case of mXDI;
b. elimination of the hydrochloric acid from the reaction medium obtained in step a) at a temperature of between 120 and 190° C. and at a pressure between 1 mbar and 20 bar.

Preferably, step b) is maintained until the assay of the resulting reaction medium indicates allophanoyl chloride content of XDI, preferably of mXDI, less than 3%, preferably less than 2%, advantageously less than 1% by weight in the reaction medium, excluding the amount of solvent, phosgene and HCl.

Step b) is a step of removing the hydrochloric acid formed during the process, which advantageously makes it possible to shift the equilibrium of reactions (1) and (2) described above to the formation of XDI. Preferably, the removal of hydrochloric acid is carried out under temperature conditions adjusted so as to avoid thermal runaway.

Step b), which promotes the conversion of intermediate chemical species, can significantly decrease the amount of chlorinated byproducts and promotes lower maintenance costs due to less fouling of the installation, which also harnesses an environmental benefit by reducing the burning of waste.

The transformation of intermediate chemical species having allophanoyl chloride functions is an indicator of the transformation of intermediate chemical species having carbamyl chloride functions. That is why the amount of allophanoyl chloride of XDI is monitored during the implementation of the process according to the invention.

The compounds of carbamyl chloride and allophanoyl chloride are compounds that release chlorides upon hydrolysis. The released chlorides, which enable the amount of carbamyl chloride and allophanoyl chloride to be determined, are obtained by reacting the reaction medium with a hot hydroalcoholic solution, according to the method of ISO standard 15028:2014. The released chlorine content was then measured by titration by any method known to the person skilled in the art, in particular by the implementation of a solution of silver nitrate.

The process according to the invention may comprise, after step b), the conventional steps of purification of the XDI obtained. In particular, the process according to the invention may comprise, after step b), a step to remove COCl₂, a step to remove the phosgenation solvent, a step to remove the heavy compounds formed during phosgenation (compounds having a strictly higher boiling point than XDI such as the dimer or trimer of XDI) and a step to remove light species formed in the phosgenation (compounds having a strictly lower boiling point than XDI such as CIBi). These steps are conventionally known to the person skilled in the art and are implemented in the conditions and with the devcies conventionally known in the art. Advantageously, to avoid any possibility of thermal runaway, step b) is carried out in a solvent and at a temperature above 120° C. and lower than 190° C.

The solvent of step b) is preferably selected from among non-reactive solvents with the isocyanate functions and in particular compatible with the conditions of implementation. Preferably, the solvent is selected from among alkanes, chloroalkanes, esters, ethers, aromatics and halogenated aromatics. Advantageously, the solvent of the step to remove hydrochloric acid may be the solvent used for the phosgenation reaction.

As mentioned above, step b) advantageously allows for the early transformation of the intermediate chemical species. Performed after the phosgenation reaction and before the subsequent steps of separating the solvent, heavy compounds and light species, this step makes it possible to work without risk of thermal runaway and degradation of products resulting from the phosgenation reaction. This transformation of intermediate chemical species before separation allows for a higher increase in the yield of total XDI.

Accordingly, it appears that the steps of separating the solvent and the heavy compounds, and the subsequent steps of separating the light species, especially the CIBi/XDI mixture, can be carried out under broader temperature and pressure ranges. Indeed, when the intermediate chemical species have been previously transformed, the thermal stability of different media is improved, allowing a favored behavior of different equipment and better productivity.

Advantageously, before step b), the reaction medium from step a) is diluted in the solvent at a rate of dilution greater than 30% by weight, preferably greater than 50% by weight. This dilution advantageously allows for a reduction in the generation of side reactions.

In a first embodiment, step b) consists of putting in contact, in a co-current, cross-current or counter-current manner, the reaction medium from step a), optionally diluted as specified above, and at least one inert compound, preferably selected from a compound of dinitrogen, argon, carbon dioxide and light alkane from C1 to C4, in a vapor or gaseous state under the operating conditions of temperature between 120° C. and 190° C., preferably between 150° C. and 190° C., and absolute pressure of between 1 and 5 bar, preferably between 1 and 3 bar. This step is implemented in a gas-liquid contactor and preferably in a stripping column.

In another embodiment, step b) is carried out by evaporation, in a batch or continuously, of hydrochloric contained in the acidic reaction medium from step a), optionally diluted as specified above, at a temperature between 25° C. and 140° C., preferably between 80° C. and 140° C. and a pressure between 10 and 1100 mbar absolute.

In another embodiment, step b) is performed by chemical sequestration of the hydrochloric acid contained in the reaction medium from step a), optionally diluted as specified above, with tertiary amines able to be immobilized on silica or on a mobile non-hydrogen base.

In another embodiment, step b) is effected by adsorption of hydrochloric acid, in particular by contacting the reaction mixture from step a), optionally diluted as specified above, with a solid substrate consisting of zeolites or ion exchange resins, particularly quaternary amine, at a temperature between 20° C. and 140° C., the hydrochloric acid remaining fixed to the solid surface and the liquid purified without hydrochloric acid.

In another embodiment, step b) is carried out by separating the reaction medium from step a), optionally diluted as specified above, by a membrane technique, in particular by pervaporation according to a specific vaporization of hydrochloric acid through an organic or ceramic membrane, with a temperature of between 30 and 160° C. and a partial pressure of hydrochloric acid in the permeate between 0.1 and 100 mbar absolute, and/or a migration by specific permeation of hydrochloric acid and retention of other species thanks to an organic or ceramic membrane at a temperature between 20 and 90° C.

In another embodiment, step b) corresponds to a distillation of the reaction medium from step a) at a temperature between 120 and 190° C., preferably between 140 and 190° C., and at a pressure between 1 and 20 bar, preferably between 12 and 17 bar. Preferably, the distillation is carried out in a column combined with a boiler and a condenser with, at the top, the removal of hydrochloric acid, and then, at the bottom, the recovery of the purified reaction medium. Preferably, the distillation takes place in batch or continuously. Preferably, the temperature of the medium in the boiler during the distillation is between 120 and 190° C., preferably between 140 and 190° C., and the pressure at the top of the distillation column is between 1 and 20 bar absolute, preferably between 12 and 17 bar absolute.

Advantageously, step b) and each separation step are performed in the presence of a polymerization inhibitor.

The final separation between the CIBi and the XDI occurs at a higher temperature, between 150 and 190° C., with a lesser pressure, between 4-5 mbar and 15 to 20 mbar, so the distillation is facilitated without risk to the safety of the installation, and the costs are reduced. Particularly advantageously, it is noted that the hydrochloric acid removal step allows a better separation of the CIBi from the XDI.

The invention also relates to a composition of XDI, particularly mXDI, in particular prepared by implementing the method defined above characterized by an XDI allophanoyl chloride content of less than 3%, preferably less than 2%, and advantageously less than 1% by weight excluding the amount of solvent, phosgene and HCl.

The invention also relates to a composition of XDI, particularly mXDI, in particular prepared by implementing the process defined above, wherein solvent residues, heavy compounds and light species are still present in combination or as a mixture after separation according to a separation process defined above with contents of less than 5%, preferably less than 3%, and advantageously less than 1.5%.

According to a particular mode of implementation, the XDI is stabilized with hindered phenols in position 2.6 such as ionols and/or a nylon-1 polymerization inhibitor, in particular during the storage period.

### PRESENTATION OF FIGURES

Other data, features and advantages of the present invention will become apparent upon reading the following detailed description of examples of non-limited embodiments, with reference to the accompanying figures, which respectively represent:

FIG. 1 shows a schematic sectional view of a column for carrying out step b) by making counter-current contact with an inert gas;

FIG. 2 shows a schematic sectional view of a membrane separation device for carrying out step b) by pervaporation;

FIG. 3 shows a schematic sectional view of a solid substrate device for carrying out step b) by fixing/absorption/selective retention of hydrochloric acid;

FIG. 4 shows a schematic sectional view of a distillation assembly comprising a column combined with a boiler and a condenser.

### DETAILED DESCRIPTION

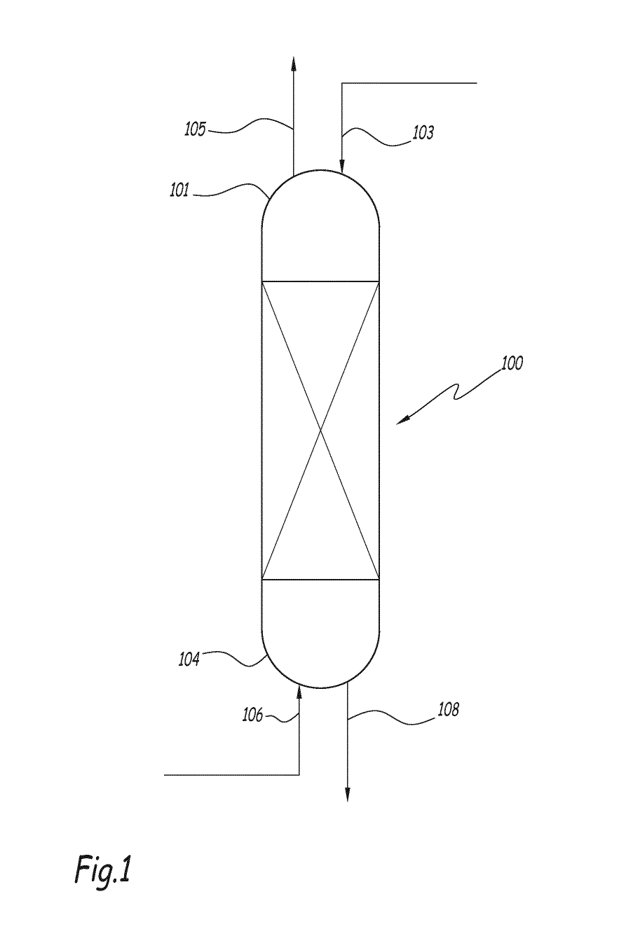

A first example of processing the reaction mixture obtained from the phosgenation is illustrated by the schematic sectional view of the column 100 of FIG. 1. The treatment here consists of making counter-current contact between the liquid mixture obtained from the phosgenation reaction and an inert nitrogen compound at 120° C. under an absolute pressure of 2 bar. This contact causes the stripping of the molecules of hydrochloric acid by transfer in the gas phase.

To do this, the reaction mixture is first diluted by adding 60% by weight of solvent before being introduced at the column top 101 through the inlet 103. The inert nitrogen compound is injected at the bottom of the column 104 through the inlet 106 in gaseous form. The hydrochloric acid emerges at the top of the column 101 with nitrogen through the outlet 105. At the bottom of the column, the mixture—purified hydrochloric acid—is recovered at the outlet 108. It has a solvent content of about 60% by weight and an XDI allophanoyl chloride content of in the order of 1% by weight excluding the amount of solvent, phosgene and HCl. The latter content is monitored by the assay described below.

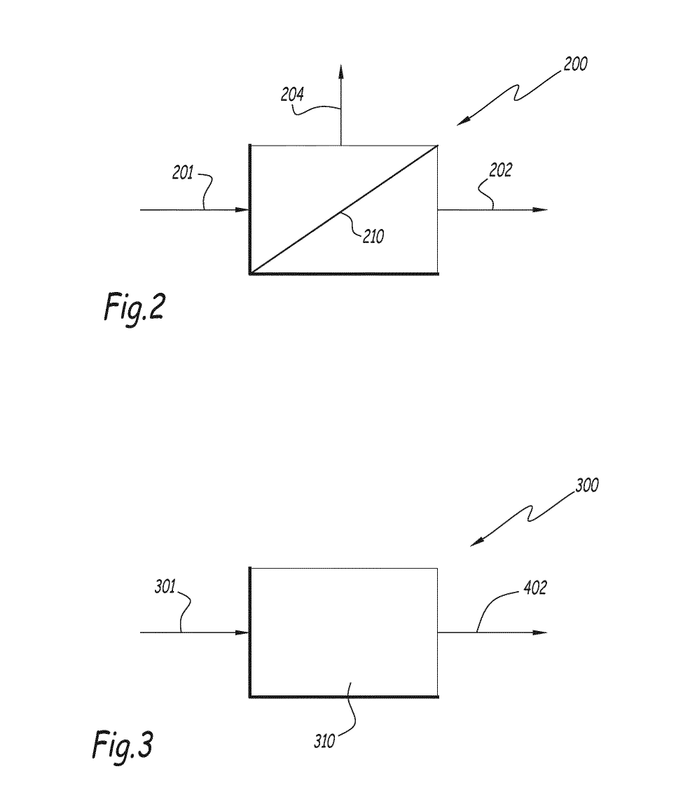

Another embodiment of step b) is illustrated by the membrane separation device 200 of the schematic sectional view of FIG. 2. The reaction mixture obtained from the phosgenation is strongly diluted by adding 60% solvent and enters said device 200 through an inlet 201. The device 200 contains a separation membrane 210, which is organic or, alternatively, ceramic.

Upon contact of this membrane 210, the hydrochloric acid is separated from said mixture by pervaporation using a specific vaporization of hydrochloric acid through the membrane 210, with a temperature of between 30 to 160° C., and partial pressure of hydrochloric acid in the permeate between 0.1 and 100 mbar absolute.

The hydrochloric acid then migrates through the membrane 210, driven by the difference in volatility for pervaporation and driven by a pressure gradient for permeation. Hydrochloric acid is then recovered at the outlet 202, while the retentate of the initial mixture—purified intermediate chemical species transformed into XDI—is recovered through the outlet 204. The retentate contains 50% by weight of solvent, and the allophanoyl chloride content is less than 2%, preferably 1% according to the control assay described below. This percentage corresponds to the weight percent of XDI allophanoyl chloride in the reaction medium by excluding the amount of solvent, phosgene and HCl.

In particular, the separation may be performed by specific permeation of the hydrochloric acid and retention of other species by an organic or ceramic membrane retention, the temperature being between 20 and 90° C., preferably at 60° C.

Another embodiment of step b) will now be given, with reference to the schematic sectional view of the solid substrate device 300 of FIG. 3. The initial mixture resulting from the phosgenation, diluted by adding solvent of 70% by weight, enters the device 300 through an inlet 301. The device 300 contains a zeolite 310 forming the solid substrate having a good affinity with hydrochloric acid. Hydrochloric acid is then adsorbed by attachment to the surface of the zeolite. The temperature is set between 20 and 140° C. at 90° C. in the example illustrated, and the solid substrate is replaced when saturated with hydrochloric acid. This type of solid substrate can be regenerated and reused.

Alternatively, the solid substrate can be replaced by ion exchange resins, where in particular tertiary or quaternary amines—preferably tertiary—can be advantageously used.

The purified mixture of hydrochloric acid is recovered at the outlet 402. This mixture has an allophanoyl chloride content in the order of 2% according to the assay monitoring implemented as set forth below. This percentage corresponds to the percent by weight of XDI allophanoyl chloride in the reaction medium at 100% of dry extract.

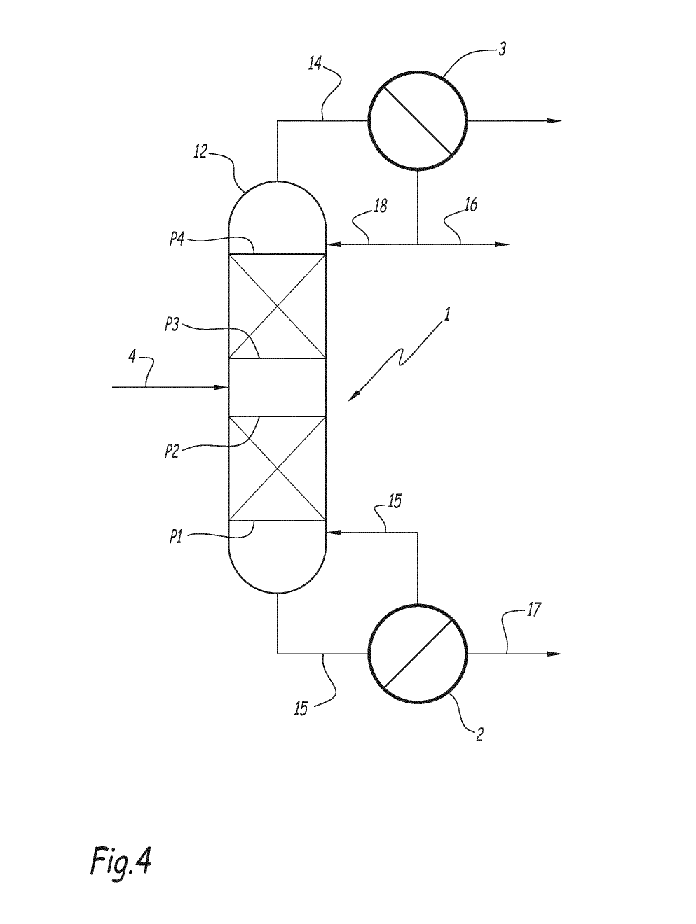

Referring to the schematic sectional view of FIG. 4, and in another embodiment of step b), a column 1 is combined with a boiler 2 and a condenser 3 for distilling the purified reaction mixture of hydrochloric acid according to any one of the preceding methods. Different plates P1 to P4 are symbolically shown at different levels of the column 1. This distillation assembly here receives a reaction mixture obtained from the phosgenation of a solution of meta-xylylene diamine or mXDA, by the power supply circuit 4 situated at an intermediate level of the column 1.

The phosgenation reaction generates the formation of intermediate chemical species, in particular chlorides of allophanoyl and carbamyl of mXDI. To perform the transformation processing of the intermediate chemical species in mXDI, the reaction medium is taken up with the solvent used for the phosgenation reaction, namely monochlorobenzene in the example illustrated. Advantageously, the solvent is added in a large amount, equal to 60% by weight of the initial mixture in the example, to prevent the generation of heavy compounds from the intermediate chemical species.

The boiler 2 is set so as to provide a temperature at the bottom of the column 12 substantially equal to 150° C. in the example illustrated. At the top of the column 12, the light products are extracted at the outlet 14: the residual hydrochloric acid and phosgene—at least partly—and part of the solvent forming, after passing through the condenser 3, the distillate 16 and the reflux 18. At the bottom of the column 12, the heavy products are recycled into recycling line 15 and the residue—purified hydrochloric acid—is recovered at the outlet 17. It contains about 60% by weight of solvent and has an XDI allophanoyl chloride content of less than 1%. This percentage corresponds to the weight percent of XDI allophanoyl chloride excluding the amount of solvent, phosgene and HCl.

According to an embodiment variant in which the remaining phosgene and the solvent are not extracted simultaneously and wherein phosgene is removed before the solvent, step b) according to the invention can be carried out during the extraction of phosgene or thereafter, but necessarily before separation from the solvent.

In the case of the simultaneous extraction of phosgene in step b), the process may be presented in this form: process for the preparation of xylylene diisocyanate XDI, particularly meta-xylylene mXDI, comprising the steps of
a) phosgenation of the xylylene diamine XDA, in particular m-xylylene diamine mXDA in the case of mXDI;
b) extraction of phosgene and simultaneous removal of the hydrochloric acid from the reaction medium obtained in step a)

In the case of the simultaneous extraction of phosgene in the removal step, the process may be presented in this form: process for the preparation of xylylene diisocyanates XDI, particularly meta-xylylene diisocyanate mXDI, comprising the steps of
a) phosgenation of the xylylene diamine XDA, in particular m-xylylene diamine mXDA in the case of mXDI, followed by a step of phosgene extraction;
b) removal of hydrochloric acid from the reaction medium obtained in step a).

In the case of processes in which the removal of phosgene occurs before or simultaneous to step b), the steps subsequent to step b) are steps involving removal of solvent and/or removal of residual light compounds and/or removal of mXDI and/or residual heavy compounds. These steps may or may not be simultaneous and may be carried out in any order.

In all processes according to the invention, it is necessary to have step b) carried out before removing the solvent.

The XDI allophanoyl chloride content is monitored by collecting a sample at the bottom of the column 12 which is assayed by gel permeation chromatography coupled to the analysis of the distribution of infrared radiation emitted, e.g. by a laser with the appropriate power.

The invention is not limited to the embodiments described and shown. For example, other solvents can be used to dilute the reaction mixture prior to step b); other inert compounds can be implemented to achieve a counter-current, cross-current or co-current stripping; feedthroughs cascaded through as a result of membrane or solid substrate devices can be developed to purify the reaction mixture of hydrochloric acid and converting the intermediate chemical species preferably into XDI; other dilution conditions; other assay methods of XDI allophanoyl chloride may be used (potentiometric assay, assay by perchloric acid, etc.).

### Exemplary Embodiments

In the examples below, the composition of the different mixtures was measured by assay after separation of the mixture on a gel filtration chromatography-type separating column (PL GEL 50 Å 60CM 7.5 MM 5 μ followed by PL GEL 50 Å 60CM 7.5 MM 5 μ) in a solvent such as dichloromethane. The detection method is infrared b measuring the NCO band at 2250 cm-1, after calibration with the mXDI of known concentration. The results of composition are given by weight, excluding the quantity of solvent, phosgene and HCl.

In some examples, the releasable chlorides (compounds having at least one allophanoyl chloride function and/or carbamyl chloride function) can be assayed by a method known as “cold chloride measurement”. This method describes the assay of releasable chlorides at a temperature between 20 and 25° C. by argentometric titration in a medium containing nitric acid, acetone and methanol. In a beaker of 100 mL, weigh a test sample containing according to the content an expected chloride:

Test sample (g)=0.071/(expected % CI)

Add 20 mL of methanol, wait 1 minute while stirring, then add 3 mL of nitric acid and 40 mL of acetone and then titrate using AgNO3 to 0.02N.

V is the volume in mL of the titrant cast for testing

T is the titre in mol/L of silver nitrate solution

E is the mass in g of the sample

M is the molar mass of the chlorine

The result of the cold chloride measurement:

Cl (mg/Kg)=(V×T×M×1000)/E

The following abbreviations have been used:

ClBi: chloromethyl-benzyl-isocyanate

mXDI: meta xylylene diisocyanate

QSF: quantity sufficient for

ARC: Accelerating Rate calorimetry

### EXAMPLE 1

In a 1L glass reactor are loaded: 351.9 g of orthodichlorobenzene and 87.4 g of crude mXDI obtained from the phosgenation of mXDA and its dephosgenation. The mixture is stirred at 300 rpm (stirring which consists of four inclined blades and baffles) and heated to 150° C. under atmospheric pressure for 5 hours under argon stripping.

The crude mXDI before step b) has the following composition by weight: 1.7% CIBi, 65% mXDI, 2% mXDI dimer, 6.7% mXDI allophanoyl chloride, 24.6% XDI-based heavy products (QSF 100).

The end product after step b) has the following composition by weight: 2.6% CIBi, 74.5% mXDI, 1.5% mXDI dimer, 0% mXDI allophanoyl chloride, 21.4% heavy products (QSF 100).

Percentage increase of mXDI following step b): ((% mXDI) after step b)t−(% mXDI) before step b))/(% mXDI) before step b)=+14.6%

### EXAMPLE 2

In a 1L glass reactor are loaded: 328.8 g of orthodichlorobenzene and 82.2 g of crude mXDI obtained from the phosgenation of mXDA and its dephosgenation. The mixture is then stirred at 300 rpm (stirring which consists of four inclined blades and baffles) and heated to 150° C. under atmospheric pressure for 5.5 hours under argon stripping.

The crude mXDI before step b) has the following composition by weight: 1.[sic]% CIBi, 68.2% mXDI, 1.5% mXDI dimer, 5.9% mXDI allophanoyl chloride, 23% heavy products (QSF 100).

The end product after step b) has the following composition by weight: 2.2% CIBi, 77.1% mXDI, 1.6% mXDI dimer, 1.3% mXDI allophanoyl chloride, 17.8% heavy products (QSF 100).

Percentage increase of mXDI following step b): ((% mXDI) after step b)−(% mXDI) before step b))/(% mXDI) before step b)=+13.0%

### EXAMPLE 3

In a 1L glass reactor are loaded: 603.4 g of orthodichlorobenzene and 216.2 g of crude mXDI obtained from the phosgenation of mXDA and its dephosgenation. The mixture is then stirred at 300 rpm (stirring which consists of four inclined blades and baffles) and heated to 130° C. under atmospheric pressure for 5.5 hours under argon stripping.

The crude mXDI before step b) has the following composition by weight: 1.6% CIBi, 75.7% mXDI, 4% mXDI dimer, 4.7% mXDI allophanoyl chloride, 14% heavy products (QSF 100).

The end product after step b) has the following composition by weight: 1.7% CIBi, 78.4% mXDI, 5% mXDI dimer, 1.7% mXDI allophanoyl chloride, 13.2% heavy products (QSF 100).

Percentage increase of mXDI following step b): ((% mXDI) after step b)−(% mXDI) before step b))/(% mXDI) before step b)=+3.6%

### EXAMPLE 4

In a 1L glass reactor are loaded: 612.3 g of orthodichlorobenzene and 202.2 g of crude mXDI obtained from the phosgenation of mXDA and its dephosgenation. The mixture is then stirred at 300 rpm (stirring which consists of four inclined blades and baffles) and heated to 130° C. under atmospheric pressure for 11 hours under argon stripping.

The crude mXDI before step b) has the following composition by weight: 1.6% CIBi, 75.7% mXDI, 4% mXDI dimer, 4.7% mXDI allophanoyl chloride, 14% heavy products (QSF 100).

The end product after step b) has the following composition by weight: 1.7% CIBi, 78.7% mXDI, 3.8% mXDI dimer, 0.9% mXDI allophanoyl chloride, 14.9% heavy products (QSF 100).

Percentage increase of mXDI following step b): ((% mXDI) after step b)−(% mXDI) before step b))/(% mXDI) before step b)=+4.0% Cold chloride measurement: before step b) 0.388% m/m, after step b): 0.058%m/m.

### EXAMPLE 5

### Thermal Stability

### Example 5a

A sample of crude mXDI, having undergone step b) according to the invention, of composition by weight (6.6% orthodichlorobenzene, 2.4% CIBi, 69.6% mXDI, 1.4% mXDI dimer, 0% mXDI allophanoyl chloride and 20% heavy products) was analyzed by ARC. 4.19 g of crude mXDI was placed in a cell of 19.7265 g, and the test was carried out according to a heat-wait-search procedure between 50° C. and 425° C. with increments every 3° C. The starting exothermic temperature of the sample is 185° C.

### Example 5b

A sample of crude mXDI of composition by weight (12.3% orthodichlorobenzene, 2.2% CIBi, 68.7% mXDI, 2.6% mXDI dimer, 3.1% mXDI allophanoyl chloride and 11% heavy products) was analyzed by ARC. 3.841 g of crude mXDI was placed in a cell of 15.2940 g, and the test was carried out according to a heat-wait-search procedure between 50° C. and 425° C. with increments every 3° C. The starting exothermic temperature of the sample is 167° C.

Comparison of Examples 5a and 5b shows that step b) has a significant impact on the starting temperature of the decomposition of the product. In fact, the starting exothermic temperature is 18° C. lower for the sample which was not treated.

1-16. (canceled)

17. A process for preparing xylylene diisocyanates XDI, in particular meta-xylylene diisocyanate mXDI, comprising the following steps:
a. phosgenation of the xylylene diamine XDA, in particular m-xylylene diamine mXDA in the case of mXDI;
b. elimination of the hydrochloric acid from the reaction medium obtained in step a) at a temperature of between 120 and 190° C. and a pressure between 1 mbar and 20 bar.

18. The process according to claim 17, wherein step b) is maintained until the assay of the resulting reaction medium indicates allophanoyl chloride content of XDI, less than 3% by weight in the reaction medium, excluding the amount of solvent, phosgene and HCl.

19. The process according to claim 17, wherein at least one solvent is added to the reaction mixture before step b), said solvent being selected from non-reactive solvents with the isocyanate functions, alkanes, chloroalkanes, esters, ethers, aromatics and halogenated aromatics.

20. The process according to claim 19, wherein the solvent added to the reaction medium is in excess of 30% by weight and advantageously greater than 50% by weight.

21. The process according to claim 17, wherein step b) is carried out in a distillation column.

22. The process according to claim 17, wherein step b) is implemented by making co-current, cross-current or counter-current contact between the reaction mixture resulting from the reaction of phosgenation and an inert compound chosen from a compound of dinitrogen, argon, carbon dioxide and light alkane from C1 to C4, at a temperature between 120° C. and 190° C., and at an absolute pressure between 1 and 5 bar.

23. The process according to claim 17, wherein step b) is carried out by distillation in a column (1) combined with a boiler (2) and a condenser (3) with, at the top of the removal (12), the residual and substantially pure hydrochloric acid, and then the removal at the bottom of the column (11) of the purified mixture.

24. The process according to claim 17, wherein step b) is carried out by evaporation, in batch or continuously, at a temperature between 25° C. and 140° C., and at a pressure between 10 and 1100 mbar absolute.

25. The process according to claim 17, wherein step b) is carried out by chemical sequestration of the hydrochloric acid by tertiary amines immobilized on silica or a mobile non-hydrogen base.

26. The process according to claim 17, wherein the step of removing hydrochloric acid is carried out by having the reaction mixture obtained from the phosgenation make contact with a solid substrate (300) consisting of zeolites (310) or ion exchange resins at a temperature between 20° C. and 140° C.

27. The process according to claim 17, wherein the hydrochloric acid removal step is performed by membrane separation (200).

28. The process according to claim 27, wherein the separation is effected by pervaporation by evaporation of the hydrochloric acid through an organic or ceramic membrane (210) at a temperature of between 30 to 160° C., and a partial pressure of hydrochloric acid in the permeate between 0.1 and 100 mbar absolute.

29. The process according to claim 27, wherein a permeation migration of the hydrochloric acid and retention of other species are performed through an organic or ceramic membrane at a temperature between 20 and 90° C.

30. The process according to claim 22 wherein step b) is carried out in batch or continuously.

31. A composition of xylylene diisocyanate XDI, particularly meta-xylylene mXDI, prepared according to claim 17, and having an allophanoyl chloride content of less than 3%.

32. The composition of xylylene diisocyanate XDI, particularly meta-xylylene diisocyanate mXDI, according to claim 31, wherein solvent residues, heavy compounds and light species are still present in combination or as a mixture after separation with content less than 5%, wherein step b) is carried out in a distillation column.

33. The process of claim 18, wherein the XDI is mXDI, and the allophanoyl chloride content of mXDI is less than 1% by weight in the reaction medium, excluding the amount of solvent, phosgene and HCl.

34. The process of claim 19, wherein the solvent is the solvent used for the phosgenation stage.

35. The process of claim 22, wherein step b) is implemented at a temperature between 150° C. and 190° C., at an absolute pressure between 1 and 3 bar.

36. The process of claim 24, wherein step b) is carried out at a temperature between 80 and 140° C.
